(19) United States
(12) Patent Application Publication
(10) Pub. No.: US 2009/0112179 A1
(43) Pub. Date: Apr. 30, 2009

(54) AUTOMATED ANIMAL DOSING SYSTEM AND METHOD

(76) Inventors: Bart Joseph Zoltan, Old Tappan, NJ (US); Aleksandr Meltser, Suffern, NY (US)

Correspondence Address: WilmerHale/Wyeth, 60 STATE STREET, BOSTON, MA, 02109, US

(21) Appl. No.: 12/067,398

(22) PCT Filed: Sep. 14, 2006
(86) PCT No.: PCT/US06/35817

§ 371 Date: Sep. 9, 2008

Related U.S. Application Data
(60) Provisional application No. 60/719,536, filed on Sep. 22, 2005.

(51) Int. Cl. A61M 5/31; A61D 7/00

(52) U.S. Cl. 604/505; 119/720; 604/65; 604/66

(57) ABSTRACT

Systems and methods for automatically delivering a dose of pharmaceutical liquid to an animal includes electronic control to automatically obtain dose information for the animal, electronic control to deliver a volume of pharmaceutical liquid to the animal in response to the dose information; and automatically recording a detailed audit trail of the liquid delivery to the animal. The dose information may be a function of a stored value of at least one physical parameter of the animal. The detailed audit trail may include, for example, information about the amount of liquid actually delivered to the animal, specifying the time of delivery, whether there were any problems with the delivery, and if so, information about such.

### CROSS-REFERENCE TO RELATED APPLICATIONS

This application claims the benefit of and priority under 35 U.S.C. §119(e) to U.S. Provisional Patent Application No. 60/719,536, filed Sep. 22, 2005 and entitled “Automated Animal Dosing System and Method,” the entire contents of which are incorporated herein by reference.

### FIELD OF THE INVENTION

This invention generally relates to systems and methods for dosing animals with pharmaceutical compositions and, more specifically, to systems and methods of automatically dosing animals with pharmaceutical compositions.

### BACKGROUND

The dosing of small animals by way of oral gavage is a common route of administration in the toxicological evaluation of potential pharmaceutical candidates and products. Oral gavage is the placement of a tube through the mouth, past the larynx, through the esophagus and into the stomach. This route is also used in in vivo discovery experiments. A defined amount of a pharmaceutical candidate, which is typically suspended or dissolved in a defined volume of an appropriate vehicle, is administered by gavage to an animal. The animal's response to the pharmaceutical candidate may then be monitored with analytical tests. Typically a large number of animals will be concurrently dosed with varying doses of the candidate, and the response of each of the animals is monitored in order to evaluate the effect of the candidate.

In this specification, the term “appropriate vehicle” is intended to mean a liquid in which a pharmaceutical candidate may be dissolved, suspended, or emulsified, and which by itself has no adverse effects. In this specification, the term “pharmaceutical liquid” is intended to refer both to the vehicle with the pharmaceutical candidate dissolved, suspended, or emulsified therein, and to the vehicle alone.

In some cases, all of the animals in the study are housed in a single room, and there are several groups of animals that receive corresponding levels of the pharmaceutical candidate. For example, there can be a low-dose group, a mid-dose group, a high-dose group, and a control (or placebo) group. It is common practice to have a “control” group of animals that receives only the “appropriate vehicle” with no drug added, to ensure that the vehicle has no measurable effects. In some cases, it is desirable to maintain an approximately equal dosing volume for each of the animals, although the amount of pharmaceutical candidate dissolved in that volume might vary according to the level of dose the animal is to receive. The dosages are most often a function of animal weight (mg/kg) and of the particular dose group the animal is in. Thus, each dose for each animal can be different.

Typically, the dose for each animal is stored on a database, and is presented to a technician on the screen of a computer terminal in communication with the database, along with the identifier of the animal to be dosed. The technician then manually prepares each unique dose, finds the appropriate animal and brings it to a dosing area, and then delivers the dose to it by way of oral gavage. However, the ability of the technician to load a syringe with a precise volume is limited, especially when doing a repeated operation over long periods of time. Further, there are no detailed records of the dosing events. For example, errors of volume reading and volume dosed cannot be independently assessed and recorded. This can result in statistical errors in the study. Also, the ability of the technician to enter information about the dosing event is limited, for example, about any anomalies that took place during dosing which might affect the outcome of the study.

### SUMMARY

The present invention provides systems and methods for automated dosing of animals.

Under one aspect, a method and system of automatically delivering a dose of pharmaceutical liquid to an animal includes an electronic control that automatically obtains dose information for the animal; an electronic control that delivers a volume of pharmaceutical liquid to the animal in response to the obtained dose information; and an electronic control that automatically records a detailed audit trail of the liquid delivery to the animal.

Under another aspect, the detailed audit trail includes information about at least one of: a time of liquid delivery, an actual volume of liquid delivered, an interruption in the delivery, an error in the delivery, an identifier for the animal, and an outcome of the delivery. The audit trail may be recorded in non-erasable memory.

Under another aspect, the dose information for the animal is a function of at least one of a concentration of the pharmaceutical liquid and a volume of the pharmaceutical liquid. The dose information for the animal may be a function of a stored value of at least one physical parameter of the animal. A value of at least one physical parameter may change over time, and electronic control may update its stored value over time. A value of at least one physical parameter may include a weight of the animal, a surface area of the animal, a value of a test result on the animal, a change in a value of a test result on the animal, and a dose group of the animal. The value of the test result may be a concentration of a chemical in at least one of the animal's urine, hair, feces, and blood. The value of the test result may be at least one of a size of a tumor of the animal and a size of an organ of the animal. The value of the test result may be a measure of cell function. The measure of cell function may be at least one of cell number, cell membrane integrity, enzyme activity, cell aggregation, and clotting function.

Under another aspect, the method includes obtaining an identifier for the animal before delivering liquid to the animal. Under another aspect, the volume of pharmaceutical liquid is delivered to the animal by one of oral gavage, nasal gavage, intravenous injection, subcutaneous injection, intramuscular injection, and peritoneal injection.

Under another aspect, electronic control automatically obtains dose information for an animal, electronic control delivers a volume of pharmaceutical liquid to the animal in response to the obtained dose information, and electronic control automatically records a detailed audit trail of the liquid delivery to the animal repeatedly for each animal of a plurality of animals, and the delivered volume of liquid is specific to each animal of the plurality of animals.

Under another aspect, electronic control loads the volume of liquid and delivers the volume to the animal. Under another aspect, electronic control loads a first volume of pharmaceutical liquid, electronic control delivers from the first volume of liquid a volume of liquid to the animal in response to the obtained dose information, and electronic control monitors an undelivered volume of liquid. Under another aspect, for each animal of a plurality of animals electronic control repeatedly and automatically obtains dose information for the animal, electronic control repeatedly delivers from an undelivered volume of liquid a volume of liquid to the animal in response to obtained dose information for the animal, electronic control repeatedly and automatically records a detailed audit trail of the liquid delivery to the animal, and electronic control repeatedly monitors an undelivered volume of liquid. Under another aspect, electronic control loads a second volume of liquid when electronic control determines whether the undelivered volume is less than the volume of liquid to deliver to a next animal.

Under another aspect, the pharmaceutical liquid is delivered with a device with a linear actuator, and a motion of the linear actuator defines the delivered volume of pharmaceutical liquid. Electronic control may use dose information to form an instruction for moving the linear actuator by a controlled amount in order to deliver the volume of pharmaceutical liquid.

Under another aspect, a method and system of automatically delivering a dose of pharmaceutical liquid to an animal includes: electronic control automatically calculating dose information for the animal, where the dose information is a dynamic function of a stored value of at least one physical parameter of the animal; and electronic control delivering a volume of pharmaceutical liquid to the animal in accordance with the dose information. Under another aspect, the method also includes, under electronic control, automatically recording a detailed audit trail of the pharmaceutical liquid delivery to the animal.

Under another aspect, a value of at least one physical parameter of the animal changes over time, and the method includes updating the stored value of the at least one physical parameter of the animal over time. Under another aspect, the method includes repeating for the animal automatically calculating dose information for the animal and delivering a volume of pharmaceutical liquid to the animal, wherein the dose information is a dynamic function of the updated stored value of the at least one physical parameter. A value of at least one physical parameter may be a function of at least one previously delivered dose of pharmaceutical liquid.

A value of at least one physical parameter may include a weight of the animal, a surface area of the animal, a value of a test result on the animal, a change in a value of a test result on the animal, and a dose group of the animal. The value of the test result may be a concentration of a compound in at least one of the animal's urine, hair, feces, and blood. The value of the test result may be at least one of a size of a tumor of the animal and a size of an organ of the animal. The value of the test result may be a measure of cell function. The measure of cell function may be at least one of cell number, cell membrane integrity, enzyme activity, cell aggregation, and clotting function.

Under another aspect, a device for delivering the volume of pharmaceutical liquid includes a linear actuator, and a motion of the linear actuator defines a delivered volume of pharmaceutical liquid. The dose information may include an instruction for moving the linear actuator by a controlled amount in order to define a controlled volume of pharmaceutical liquid.

Under another aspect, a system for automatically delivering a dose of pharmaceutical liquid to an animal includes: storage for storing dose information for the animal and a detailed audit trail of delivery of liquid to the animal; an electronically-controllable dosing device responsive to controller commands for delivering a volume of pharmaceutical liquid to an animal; and a controller in communication with the storage device and with the dosing device. The controller includes electronic logic to: access the storage to receive dose information; automatically determine the volume of pharmaceutical liquid to deliver to the animal from the dose information; automatically send controller commands to the dosing device; and automatically send a detailed audit trail to the storage for storage.

Under another aspect, the system includes electronic logic to automatically determine the dose information using a value of at least one physical parameter of the animal. A value of at least one physical parameter of the animal may include a weight of the animal, a surface area of the animal, a value of a test result on the animal, a change in a value of a test result on the animal, and a dose group of the animal.

Under another aspect, the storage includes non-erasable memory. The storage may be remote to the controller. The storage may be co-located with at least one of the controller and the dosing device. The storage may be one of a mainframe computer and a personal computer. The storage may be an RFID chip associated with the animal. The RFID chip may be implanted in the animal.

Under another aspect, the dosing device includes an automatic syringe. Under another aspect, the dosing device includes one of an electronically controllable peristaltic pump and an electronically controllable positive displacement pump.

Under another aspect, the dosing device includes an electronically controllable linear actuator assembly. The controller may include one of electronic logic and embedded software for determining a travel distance for a linear actuator based on the dose information. The travel distance may be directly related to the volume of pharmaceutical liquid delivered.

Under another aspect, the dosing device includes electronic circuitry to receive and respond to controller commands and to send one or more delivery signals to the controller. The controller may include one of electronic logic and embedded software to monitor the one or more delivery signals from the dosing device and to construct an audit trail using the one or more delivery signals. The audit trail may include at least one of a time of pharmaceutical liquid delivery, an actual volume of pharmaceutical liquid delivered, an interruption in the delivery, an error in the delivery, an identifier for the animal, and an outcome of the delivery.

Under another aspect, the dosing device includes an input device through which a user can input at least one of the following commands to control the system: request dose information for an animal, load a volume of pharmaceutical liquid into the dosing device, and deliver a volume of pharmaceutical liquid from the dosing device to the animal. The controller may include a display to provide at least one of the following pieces of information to a user: a unique animal identifier, indication that the dosing device is ready to load the volume of pharmaceutical liquid, and that the dosing device has loaded the volume of pharmaceutical liquid.

Under another aspect, the system includes electronic logic responsive to controller commands to: control loading of the pharmaceutical liquid into the dosing device, control delivery of the pharmaceutical liquid to the animal from the dosing device, and to automatically monitor one or more volumes of pharmaceutical liquid loaded into and delivered from the dosing device. The electronic logic may respond to controller commands by loading a first volume of pharmaceutical liquid into the dosing device, by delivering from said first volume of liquid a volume of liquid to a first animal in accordance with dose information for said first animal, and by monitoring a first undelivered volume of pharmaceutical liquid. The electronic logic may respond to controller commands by delivering from said undelivered volume of pharmaceutical liquid a volume of pharmaceutical liquid to a second animal in accordance with dose information for said second animal, and by monitoring a second undelivered volume of pharmaceutical liquid.

Under another aspect, the controller is a wearable device.

Under another aspect, the system further includes an animal identifier. The identifier may be one of a number, a bar code, and an RFID tag associated with the animal. The dosing device may further include a reader for the animal identifier.

Under another aspect, the storage device and controller communicate by one of a wired and a wireless communication link.

### BRIEF DESCRIPTION OF THE DRAWINGS

In the Drawings:

FIG. 1 is a flow chart of steps in a method for automatically dosing rodents according to one embodiment of the invention.

FIG. 2 is a block diagram of a system for automatically dosing rodents according to one embodiment of the invention.

FIG. 3 is an illustration of an automatic syringe that can be used as part of a system for automatically dosing rodents according to one embodiment of the invention.

FIG. 4A is a block diagram of a system for automatically dosing rodents according to one embodiment of the invention.

FIG. 4B is a flow chart of steps in the operation of the system of FIG. 4A according to one embodiment of the invention.

FIG. 5A is a block diagram of a system for automatically dosing rodents according to one embodiment of the invention.

FIG. 5B is a flow chart of steps in the operation of the system of FIG. 5A according to one embodiment of the invention.

### DETAILED DESCRIPTION

Preferred methods and systems for automated dosing of animals automatically deliver a predefined amount of a pharmaceutical composition to an animal. The methods and systems generally, under electronic control, automatically obtain dosing data for a particular animal, and use that data to determine an appropriate dose volume for that rodent, and then deliver that precise volume to the animal. In some cases, the volume is delivered using a linear actuator assembly. The motion of the linear actuator assembly translates to a defined volume of liquid.

In general, a large number of animals can be accurately dosed with preferred systems and methods for automated dosing, even though the volume of liquid to be delivered may be specific to each animal. The dose volume may be a function of one or more parameters or variables, at least some of which can be continuously updated. For example, the dose volume could be a function of a weight, a surface area, and/or a continually updated test result of the animal. The methods and systems can easily incorporate newly obtained information into the determination of appropriate dose volumes for animal. In general, a large volume of liquid can be loaded into the dosing device, and different portions of the volume can be delivered to different animals in accordance with their appropriate dose volumes; or a volume of liquid for an individual animal can be loaded and then delivered to that animal.

The methods and systems can also record an accurate audit trail, or delivery history, for the amount of liquid delivered to the animal, for example, specifying the time of delivery, the amount of pharmaceutical liquid actually delivered, whether there were any problems with the delivery, and if so, information about such. In some methods and systems, the protocol data and audit trail are stored on a remote database, which may be a mainframe computer. In other described methods and systems, the protocol data and audit trail are stored co-locally to a controller or dosing device. In still other described methods and systems, the protocol data and audit trail are stored in an RFID chip affixed to, e.g., implanted in, the animal itself. Then it may not be necessary to access a remote database, because all of the necessary information is stored with the animal.

Dosing an animal is generally intended to mean administering or delivering a predefined amount of a pharmaceutical composition. The animal may be one of a plurality of animal, each of which can be dosed with the same or with a different pharmaceutical liquid. The system can be used, for example, to study the effects of the pharmaceutical liquid on over 1000, or over 2000, or even a larger number of animals.

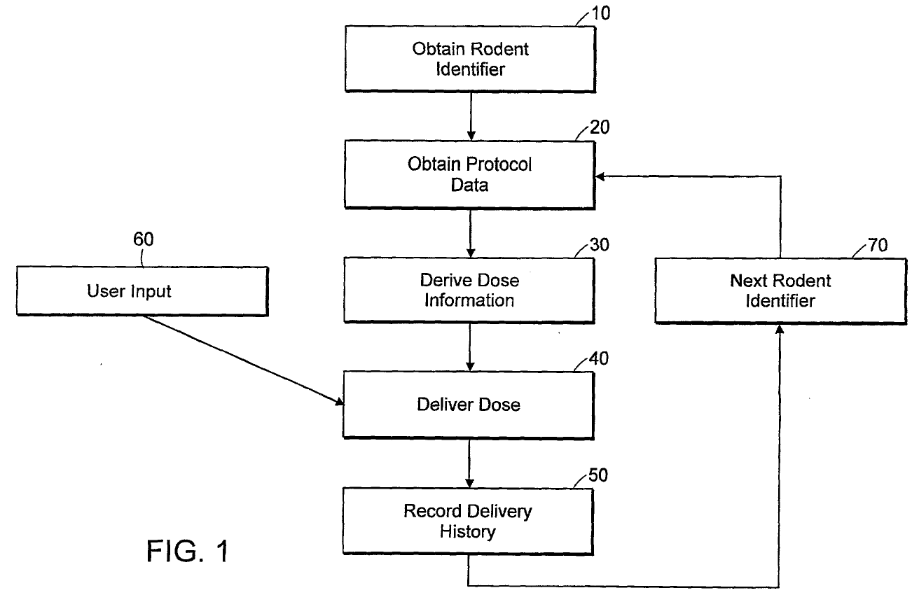

FIG. 1 is a flow chart of steps for a method of automatically delivering a dose of a pharmaceutical liquid to each animal of a plurality of animals, e.g., each rodent of a plurality of rodents, according to one embodiment of the invention. Each rodent has an associated identifier, which allows it to be uniquely identified among other rodents, as described in greater detail below. First, an identifier for a first rodent is obtained (step 10).

Next, the rodent identifier is automatically used, under electronic control, to obtain protocol data relating to the dose (step 20). The protocol data is stored electronically, and includes information related to the volume of the pharmaceutical liquid that should be delivered to the rodent. The protocol data may also include, for example, one or more physical parameters of the rodent, e.g., the weight and/or the surface area of the rodent, and/or the concentration of the liquid to be delivered to the rodent. The dose defined in the protocol may also be flexibly calculated based on the values of one or more parameters, for example, the results of analytical tests on the rodent. In some cases, the protocol data may include the identifier of the rodent that is to be dosed, in which case the protocol data and identifier are obtained concurrently.

Next, under electronic control, the protocol data is used to determine dose information about the volume of pharmaceutical liquid to deliver to the rodent (step 30). The dose information may be a function of at least some of the protocol data. For example, if the protocol data says that the rodent should receive 10 mg of a drug that is suspended in the pharmaceutical liquid per gram of rodent weight, then the dose information can be determined by calculating what volume of liquid should be delivered to the rodent based on the rodent weight and the concentration of the drug in the liquid. In general, the volume of liquid will be specific to each rodent of a plurality of rodents.

Next, a user inputs a confirmation that the rodent is prepared for liquid delivery (step 60). In general, the user has a limited involvement in the process of dosing the rodent, except for confirming that the rodent is prepared for dosing. Examples of ways that the user might confirm the rodent is prepared for dosing are described in greater detail below.

When the user input is received, then the proper dose of the pharmaceutical liquid is delivered to the rodent, under electronic control (step 40), based on the derived dose information (step 30). Examples of systems for delivering pharmaceutical liquids to rodents are described in greater detail below.

Next, the history of the delivery, or audit trail, is recorded (step 50), under electronic control. The history can include, for example, information about whether the dose was successfully delivered, an actual volume of liquid that was delivered, whether there were any delivery problems, how long they persisted, and how they were corrected, an identifier for the rodent, and the time at which the dose was delivered. Recording delivery history is useful because it allows a detailed record, or audit trail, to be automatically kept over time of the amounts of pharmaceutical liquid administered to the rodent. This can be useful in ensuring that all rodents receive the intended dose of liquid at the correct time, and reduces the possibility of human error resulting in incorrect record keeping. This can also be useful, for example, in combination with other information such as the results of tests on the rodent in order to determine the effect of the pharmaceutical liquid on the rodent. In some embodiments the step of recording is a “write-once” process, for example, the audit trail is recorded in non-erasable memory. In other words, once the history is automatically recorded, it cannot be altered. This can be useful when presenting the results of a study to a regulatory agency, because the possibility of the manipulation of delivery data is eliminated.

Steps 10 through 60 correspond to steps that are executed for a first rodent. If another dose of a pharmaceutical liquid is to be delivered to a next rodent, then the identifier for that rodent is obtained (step 70). Then, previously described steps 20-60 of the method are executed for that next rodent.

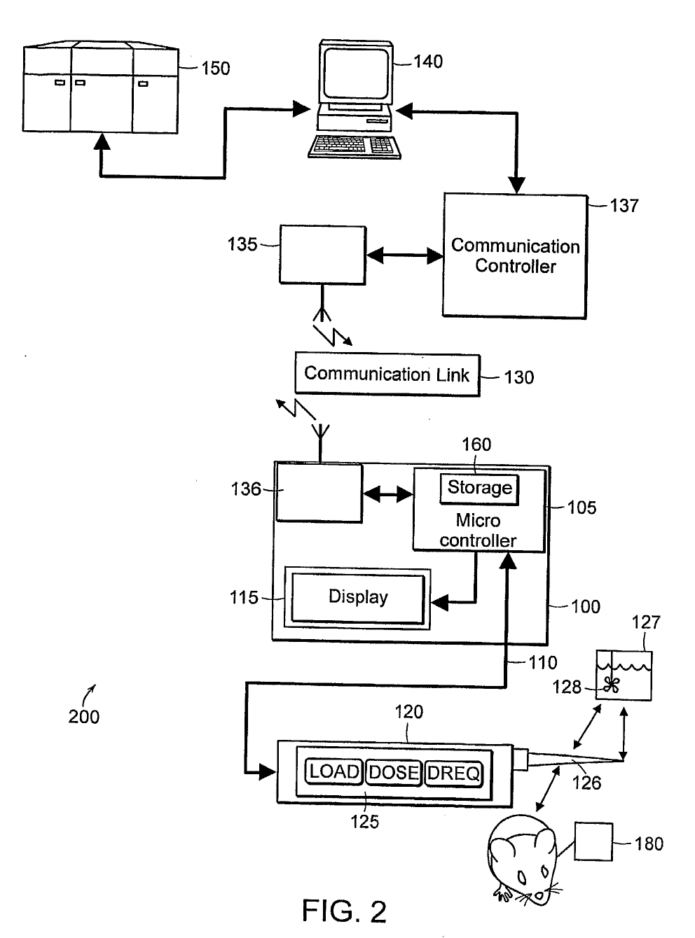

FIG. 2 illustrates an embodiment of a system for automatically delivering a dose of a pharmaceutical liquid to each rodent of a plurality of rodents, according to the method illustrated in FIG. 1. The system can be used, for example, to conduct toxicological studies on each rodent in the plurality of rodents. The system can be flexibly programmed to dose rodents on the basis of test results, which may be continuously updated. The system also maintains a detailed history, or audit trail, of the delivery of liquid to each rodent.

System 200 includes rodent identifier 180, database 150 that contains protocol data associated with identifier 180, automatic dosing device controller 100, and automatic dosing device 120. In some embodiments, storage device, e.g., database 150 instructs dosing device controller 100 as to which rodent, identified by identifier 180, should be dosed. Either controller 100 or database 150 calculates or determines dose information for the rodent from the protocol data. Controller 100 sends the dose information to dosing device 120, which obtains and delivers the determined amount of pharmaceutical liquid to the rodent, with limited assistance from a user (not shown). After the liquid is delivered, controller 100 sends an audit trail, or delivery history, to database 150, which stores the history.

Each rodent of the plurality of rodents has an associated unique rodent identifier 180. Database 150 stores identifier 180 for each rodent, along with relevant information associated with each rodent as described in greater detail below. Rodent identifier 180 can generally be any value that is conveniently stored on and recalled from database 150, and that is sufficient to uniquely identify each rodent of the plurality of rodents. For example, in one embodiment identifier 180 is a number of a specified length, which could be tattooed on the rodent, or printed on a tag attached to the rodent or to the rodent cage.

Database 150 stores the value of rodent identifier 180 for each rodent, along with other relevant information about each associated rodent, as well as protocol data related to doses to be delivered to each rodent. This information may be different from rodent to rodent, but because the system is automated the doses may be easily tailored to each rodent. Database 150 generally contains information about the study being performed on each rodent. Database 150 has the functionality of recalling and storing information in response to commands that are input from database terminal 140. Information about a particular rodent can be recalled from database 150 by inputting the corresponding unique rodent identifier 180 to database terminal 140. Database 150 also may include electronic logic for determining protocol data and/or dose information using one or more variables or parameters of the animal. In one embodiment, database 150 is a VAX mainframe computer running database software and storing a database of information, and database terminal 140 is a computer, e.g., PC, running software suitable for communicating with database 150 and for calculating protocol data and/or dose information. Xybion is an example of a toxicology database that can be used in this embodiment. In another embodiment, database terminal 140 is a computer, e.g., PC, and database 150 is software running on the terminal that accesses stored information on a hard drive attached to the computer.

Database 150 stores, for example, one or more physical parameters for each rodent, such as the rodent size information. The size information may include the weight and/or the surface area of each rodent. In some embodiments, database 150 obtains this information through one or more measurement devices that are directly linked to the database. For example, the rodent can be placed on a scale that is directly linked to database 150, and database 150 can directly record the rodent weight. This is useful because it reduces the possibility of human error resulting in an incorrect weight value being input to database 150. Another physical parameter includes a dose group to which the rodent belongs, for example a low-dose, mid-dose, high-dose, or placebo group. Typically the volume of pharmaceutical liquid depends on the dose group of the rodent in addition to other parameters.

Database 150 also stores an audit trail, or dose delivery history for each rodent, which is described in greater detail below.

Database 150 also stores information regarding the pharmaceutical liquid or liquids to be dosed to each rodent. This information could include, for example, the concentration, and/or the viscosity of the liquid. Typically, the composition is a pharmaceutical liquid that contains a certain concentration of a suspended or dissolved drug. For example the concentration may have units of mg of drug per ml of solution. It could also include instructions about mixing or aspirating the liquid prior to delivery.

Database 150 also stores protocol data related to the particular study in which the rodent is involved. In general, the identity of the pharmaceutical liquid and the volume and/or concentration of that liquid to be delivered can vary from rodent to rodent and even over time for the same rodent. In one embodiment, the protocol data includes the weight of the rodent, the concentration of the liquid, and the weight of a drug suspended/dissolved in that liquid that should be delivered to the rodent per unit of rodent weight. This information can be used together to determine the amount of liquid to be delivered, e.g., the dose information. In another embodiment, the protocol data includes the surface area of the rodent, the concentration of the liquid, and the weight of a drug suspended/dissolved in the liquid that should be delivered to the rodent per unit of rodent surface area. These examples of protocol data are only intended to be illustrative of data that can be used together to determine a dose for the rodent. In general, any data can be used that is appropriate to determine dose information for a pharmaceutical composition to be delivered to a particular rodent.

Database 150 also stores other relevant physical parameters about each rodent, for example a continuously updated history of analytical test results on the rodent. For example, the history could include the results of measurements over time on different parameters of the rodent that might be affected by the presence of the pharmaceutical liquid. For example, the physical parameters might include test results on the concentration of a chemical in, the rodent's blood, serum, urine, hair, and/or feces. The history could include test results on the size of one or more of the rodent's organ, such as the liver, and/or of a tumor. The history could also include necropsy results after sacrifice of the rodent. In some embodiments, the database combines the history of analytical test results with the dosing history to derive conclusions about the toxicology of a delivered pharmaceutical liquid. The values of one or more of these parameters may depend in part on previously delivered volumes of a pharmaceutical liquid, e.g., are dependent variables. The values of these parameters/variables may be continuously monitored, and continuously updated.

The values of one or more of these physical parameters can automatically be used to determine the required dose information via the protocol data, under electronic control. In other words, the database can use a flexible, reprogrammable, algorithm that can dynamically calculate parameter-dependent protocol data and/or dose information, taking as input at least one parameter. One or more of the parameters, e.g., test results, themselves may be dosage dependent, e.g., depend on earlier doses of liquid pharmaceutical compositions that were delivered to the rodent. Further still, the database could use changes in the values of the test results, as opposed to the values themselves, in part to determine protocol data and/or dose information. For example, in the treatment of hyponaturemia, the rate of change of sodium in the rodent's serum determines in part how much of a drug may safely be given. Because database 150 contains continuously updated results of analytical tests, dosing a rodent with a volume that is based on the latest rate of change of a measured parameter, such as sodium, is feasible. In general, the system can automatically and accurately dose a rodent based on any information that may be relevant to a particular study on the rodent. In general, the appropriate volume of liquid will be specific to each rodent of a plurality of rodents.

Database 150 optionally determines dose information from the protocol data, and transmits that information to dosing device controller 100. Otherwise, database 150 transmits the protocol data to controller 100, and controller 100 then uses that data to determine the dose information.

Dosing device controller 100 obtains either the protocol data or the dose information from database 150 via first communication link 130. More specifically, dosing device controller 100 and database terminal 140 each are in communication with, or include integrated respective communication hardware 136 and 135. Hardware 136 and 135 communicate via link 130. In some embodiments, hardware 136 and 135 are wireless modems, e.g., Bluetooth modems, and link 130 is a bidirectional wireless communications link. Optional communication controller 137 assists with communication between dosing device controller 100 and database terminal 140 via path 136, 130, 135. For example, information that controller 100 sends via path 136, 130, 135 might not be directly understandable by database terminal 140, and information that database terminal sends via the path might not be directly understandable by controller 100. Communication controller 137 has the functionality of making information sent between controller 100 and database terminal 140 understandable to the other device. In one embodiment, communication controller 137 is a keyboard emulator that emulates keystroke entries into database terminal 140. In another embodiment, communication controller 137 is a code-operated switch that assists communication between controller 100 and terminal 140. In general, link 130 can be any kind of link including suitable hardware and/or software that enable dosing device controller 100 and database terminal 140 to communicate information to one another.

Dosing device controller 100 includes electronic logic, e.g., microcontroller 105. Microcontroller 105 has the functionality of accessing, e.g., sending information to and receiving information from database terminal 140 via path 136, 130, 135; of automatically determining the volume of pharmaceutical liquid from information provided by database terminal 140; of sending controller commands to dosing device 120 as described in greater detail below; and automatically sending a detailed audit trail to database terminal 140 for storage on database 150. Microcontroller 105 includes controller storage medium 160, which may locally store rodent identifier 180, protocol data, dose information, and/or delivery history, as described in greater detail below. Dosing device controller 100 may also include embedded software which performs some or all of the functions of microcontroller 105.

Dosing device controller 100 further includes display 115 through which it provides relevant information to a user. As discussed in greater detail below, the operation of the device is regulated to a limited extent by commands issued by the user in response to information provided by display 115. For example, in some embodiments protocol data on database 150 dictates which rodent is to be dosed. Display 115 then displays identifier 180 for the rodent to be dosed, so that the user can find the rodent and prepare it for dosing, and confirm the preparation with a command. In one embodiment, display 115 is an LCD display, although any suitable kind of display for communicating appropriate information to the user can be used.

In one embodiment, dosing device controller 100 is a small, wearable, battery-powered device that the user can wear, for example, on a belt. This is useful because the user can freely move around the room and dose rodents without needing to return to a central location to receive information. In another embodiment, dosing device controller 100 is a computer, e.g., a PC. In general, dosing device controller 100 can be any device that can use information provided by database terminal 140 to then provide dose information to dosing device 120.

When controller 100 obtains dose information for a particular rodent, controller 100 sends controller commands in accordance with the dose information to dosing device 120 via second communications link 110, which in some embodiments is bidirectional. Then dosing device 120 loads the dose according to the controller commands, e.g., received dose information, and later delivers the dose to the rodent, with assistance from the user as described in greater detail below. In one embodiment, dosing device 120 is physically tethered to dosing device controller 100, and link 10 is a wired link such as a serial connection. In general any suitable wired or wireless communication link 110 between dosing device controller 100 and dosing device 120 can be used.

Dosing device 120 is preferably an electronically controlled, automated, hand-held device that is easily manipulated by the user, and that is similar in some aspects to manual dosing devices. For example, a user may be used to using a manual device to dose rodents, and so it may be preferable that dosing device 120 has a similar look and feel to a manual device in order to make it easier for the user to use the device. Also, some manual dosing devices are currently approved by regulatory agencies for use in the delivery of pharmaceutical liquids to rodents. For example, manual devices may be made of a certain kind of approved plastic that does not react with a liquid during a typical residence time of the liquid, and it is preferable that the components of automatic dosing device 120 that contact the liquid are also made of an approved plastic. It is preferable that dosing device 120 has at least the characteristics that regulatory agencies require for the approval of manual devices.

Dosing device 120, which may be an automatic syringe, includes gavage tip 126, through which pharmaceutical liquid is loaded (aspirated) and/or delivered with assistance from the user. Gavage tip 126 can be disposable plastic, though any suitable tip can be used. Dosing device 120 also includes keypad 125, through which the user inputs simple commands to system 200. In part, input from the user on keypad 125 regulates communication between dosing device controller 100, database terminal 140, and dosing device 120, as described in greater detail below. For example by pressing the “DREQ” button on keypad 125, the user signals to controller 100 to obtain dose information for a rodent, from database terminal 140. Operation of other buttons is described in greater detail below.

With assistance from the user, dosing device 120 loads a defined volume of pharmaceutical liquid from liquid source 127, which optionally is agitated or stirred by stirring device 128. Specifically, the user inserts gavage tip 126 into liquid source 127, and presses a button on keypad 125 of dosing device 120 that causes dosing device 120 to load or aspirate the calculated amount of liquid. Liquid source 127 optionally includes many different liquids (not shown), from which one particular liquid can be automatically loaded by dosing device 120 in response to the dose information. Methods for loading pharmaceutical liquid into dosing device 120 are described in greater detail below.

In one embodiment, dosing device 120 loads, or aspirates, a relatively large volume of pharmaceutical liquid from liquid source 127. Then device 120 automatically delivers an appropriate aliquot of the liquid to each rodent. This embodiment can be used, for example, with a homogeneous liquid, e.g., a liquid that has a uniform concentration of drug throughout its volume. In another embodiment, dosing device 120 is a relatively low volume, high-speed dosing device. In this case dosing device 120 allows many rodents to be dosed in rapid sequence by automatically loading, or aspirating, and holding an appropriate volume of pharmaceutical liquid from liquid source 127 for each individual rodent. Then dosing device 120 delivers that volume. This embodiment can be used with most liquids, and is particularly useful if the pharmaceutical liquid is a suspension of a drug in a solvent, in which case the drug might separate out of the solvent over time. In this case liquid source 127 optionally includes stirrer 128, e.g., a stir bar, to keep the drug homogeneously suspended in the solvent, so that when dosing device 120 loads a volume of the liquid it is substantially homogeneous.

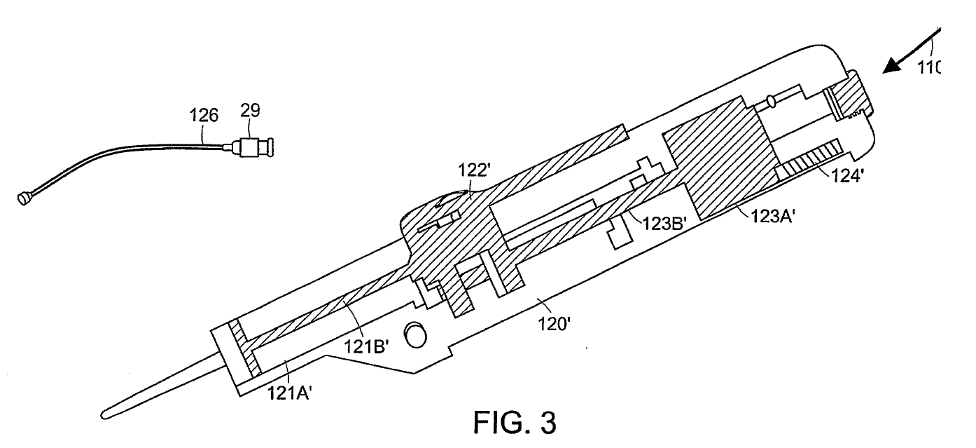

One embodiment of a dosing device, e.g., automatic syringe, which can be used with system 200, is illustrated in FIG. 3. Dosing device 120′ includes a syringe having a barrel 121A′ and a plunger 121B′. Gavage tip 126′ (inset) attaches to syringe barrel 121A′. Gavage tips are commercially available, for example from Popper and Sons. Dosing device also includes plunger lock mechanism 122′, electronically controllable linear actuator assembly 123A′, 123B′, and motion control and interface electronics 124′. Dosing device 120′ communicates with dosing device controller (not shown in FIG. 3) via communication link 110′. Dosing device controller (not shown) includes electronic logic and/or embedded software for a travel distance to move the linear actuator using the dose information. The travel distance is directly related to the volume of pharmaceutical liquid to be delivered to the rodent.

Dosing device 120′ holds syringe barrel 121A′ in a fixed position. Syringe plunger 121B′ has a proximal end (not visible in this view) that is locked into plunger lock mechanism 122′. Barrel 121A′ and plunger 121B′ can be made of any suitable material, such as plastic or glass, and may be disposable. Disposable plastic syringes are commercially available, for example from Plastibrand®. In some embodiments, the syringe has a volume between 1 and 20 milliliters, for example. In general, it may be preferable to use relatively small volume syringes to dose rodents with individual amounts of liquid, and relatively large volume syringes to dose multiple rodents with aliquots of a volume of liquid. In some embodiments, barrel 121A′ and plunger 121B′ are disposed of after liquid is delivered to each rodent in a particular dosage group of a study, e.g., a low-dose group, a mid-dose group, a high-dose group, or a placebo group.

It may be preferable in some embodiments to dose each rodent with an individual amount of liquid that is prepared immediately before delivering the liquid to the rodent. This can reduce the residence time of the liquid inside of dosing device 120′. In some embodiments, the residence time is 30 seconds or less. This is useful because it limits the time over which any reaction between the liquid and the device might take place, which may, for example, enhance the accuracy of a toxicology study.

In one embodiment, linear actuator assembly 123A′, 123B′ is a stepper-motor driven linear actuator including stepper motor 123A′ and motor shaft 123B′. Motion control and interface electronics 124′ send signals that cause the stepping or actuation of stepper motor 123A′ in one of two selected rotational directions, with a selected step resolution, e.g., number of steps per rotation, for a selected period of time. For example, the electronics may be directly controlled by input from the user on keypad 125 (not shown in this view). For example, if the user presses a “LOAD” key, the motor might step in one direction, and if the user presses a “DOSE” key, the motor might step in the opposite direction.

Plunger lock mechanism 122′ is movably connected to motor shaft 123B′. When motor 123A′ steps in a given rotational direction, motor shaft 123′ rotates in a corresponding rotational direction, causing plunger lock mechanism 122′ moves in a corresponding linear direction. The linear motion of mechanism 122′ moves locked plunger 121B′ through syringe barrel 121A′. If motor 123A′ linearly moves the flat, distal end of plunger 121B′ up and to the right, in this view, plunger 121B′ draws a vacuum in syringe barrel 121A′. For example, if gavage tip 126′ is inserted in a liquid, then this motion will load the liquid into barrel 121A′. If motor 123A′ linearly moves the flat, distal end of plunger 121B′ down and to the left, in this view, plunger 121B′ pushes out the contents of syringe barrel 121A′. For example, if barrel 121A′ contains a liquid and gavage tip 126′ is inserted into the stomach of a rodent, then this motion will deliver the liquid to the rodent.

In general, a motion of the linear actuator assembly 123A′, 123B′ defines the delivered volume of pharmaceutical liquid, and the controller sends an instruction to dosing device 120′ to move the linear actuator by a controlled amount in order to define a controlled volume of liquid. Specifically, the volume of liquid that dosing device 120′ loads and delivers to the rodent is related to the distance over which the plunger is moved, e.g., the stroke length of assembly 123A′, 123B′, and to the volume of syringe barrel 121A′. If syringe barrel 121A′ has a relatively small diameter, e.g., is a 1 ml syringe, then a given stroke length will load and deliver a relatively small volume of liquid. If syringe barrel 121A′ has a relatively large diameter, e.g., is a 20 ml syringe, then that same stroke length will load and deliver a relatively large volume of liquid. In general, errors in the loaded and delivered volume will depend linearly on the stroke length. In some embodiments, the stepper motor enables dosing device 120′ to load a volume of liquid with high accuracy, which is repeatable over a large number of delivery events. For example, each step of the motor may correspond to 1 microliter of liquid or less.

In other embodiments, dosing device 120′ may include a different mechanical component than linear actuator assembly 123A′, 123B′ that has the functionality of drawing liquid into and delivering liquid from syringe barrel 121A′. For example dosing device 120′ may include a high-accuracy, electronically controllable, reversible peristaltic pump, or a high-accuracy, electronically controllable, reversible positive displacement pump. In this case, the liquid may reside in pump tubing for a prolonged period of time, so the tubing should be selected to minimize reactivity with the liquid over at least the residence time. It would also be preferable to dispose of and replace the after liquid is delivered to each rodent in a particular dosage group of a study, e.g., a low-dose group, a mid-dose group, a high-dose group, or a placebo group.

In general, referring to FIG. 2, dosing device 120 can be any device that has the functionality of loading, or aspirating, a predetermined amount of pharmaceutical liquid into the device in response to dose information provided by dosing device controller 100. Dosing device 120 has the functionality of delivering the predetermined amount of pharmaceutical liquid, defined by the dose information, to a rodent. Dosing device 120 includes electronic circuitry or logic to receive and respond to controller commands. Dosing device 120 also includes logic to send one or more delivery signals to controller 100 that enable controller 100 to construct an audit trail, or delivery history, of the delivery of liquid to the rodent. Controller 100 monitors the one or more delivery signals from dosing device 120 and from these signals constructs an audit trail. Controller 100 then sends the audit trail to database 150. In one embodiment, controller 100 and dosing device 120 are physically integrated, and can be considered to be a single device that has the described functionality of both controller 100 and dosing device 120.

First communication link 130 between dosing device controller 100 and database terminal 140, and second communication link 110 between dosing device controller 100 and dosing device 120 can each be one of many kinds of communication links. For example, one or more of links 110, 130 can be wireless, and/or one or more of links 110, 130 can be wired. Examples of wireless communication links include radio links, IR links, and Bluetooth links, and wireless internet links. Examples of wired communication links include Ethernet, USB, and RS232 (serial) links. In one or more embodiments, one or more of links 110, 130 could be other kinds of communication links, e.g. unidirectional links. In general, links 110 and 130 each have the functionality of communicating relevant information between the appropriate components of system 200.

In general, the user's interaction with system 200 is limited, and the user's physical interactions with a rodent are similar to the interactions in manual rodent dosing, e.g., obtaining a rodent, inserting the gavage tip into the pharmaceutical liquid to aspirate the liquid, and inserting the gavage tip into the stomach of the rodent to deliver the liquid. In this case, however, the rodent is automatically identified, the appropriate volume of liquid is automatically aspirated and delivered, and an audit trail is automatically recorded for the delivery.

First, the user presses the “DREQ” button on keypad 127 of dosing device 120, which is in communication with controller 100 via second communication link 110. The “DREQ” command, which stands for “Data Request,” signals to controller 100 to request protocol data for a rodent from database 150. Other buttons or commands that are suitable to initiating a request for protocol data can be used instead of “DREQ.” At this point, the user does not yet know which rodent is to be dosed.

Then, controller 100 automatically sends a request for protocol data for a rodent to database 150. Specifically, controller 100 transmits the request signal from communication hardware 136, via communication link 130, to communication hardware 135, and optionally then to communication controller 137. As described above, communication controller 137 may assist in the interpretation of data between database 150 and controller 100. Then, the request signal is transmitted to database terminal 140, which automatically obtains protocol data from database 150 for a rodent associated with identifier 180. The particular rodent that database 150 selects might be based on a protocol of the study.

Then, in one embodiment, database terminal 140 sends the protocol data to controller 100 via optional communication controller 137, communication hardware 135, communication link 130, and communication hardware 136. Controller 100 then uses that data to derive dose information. In another embodiment, database 140 derives dose information from the protocol data and sends the dose information to controller 100 via the described path. A possible format for sending the protocol data and/or dose information is described in greater detail below.

Controller 100 displays at least a portion of the protocol data and/or dose information on display 115, including identifier 180 of the rodent to be dosed. The user did not previously know which rodent is to be dosed, and so the user then finds and obtains that rodent according to the displayed identifier 180. Optionally, the user finds the appropriate rodent with the assistance of information provided by database 150, which controller displays on display 115. For example, the information may include information about a physical location of the rodent, such as a particular group of rodents that the rodent is kept with, and/or a cage number of the rodent. This sort of information may be stored systematically on database 150 along with other rodent information, such as the rodent size and/or results of analytical measurements on the rodent.

When controller 100 obtains the dose information, either by deriving it or by receiving it directly from database terminal 140, controller 100 automatically sends the dose information to dosing device 120 via second link 110. In some embodiments, controller may reformat the dose information before sending it to dosing device 120. This can be useful, for example, if the dose information provided by database terminal 140 is in a format that dosing device 120 cannot interpret. In this case, dosing device controller 100 would convert the dose information into a format that can be appropriately used by dosing device 120 to deliver an appropriate amount of pharmaceutical liquid to the rodent.

When controller 100 sends dose information in an appropriate format to dosing device 120, controller 100 displays “ready to load liquid” or another appropriate signal to the user on display 115, which lets the user know that the dose information is ready. Controller 100 also optionally displays the identity and/or concentration of the liquid in liquid source 127 to be delivered to the rodent. The user then inserts gavage tip 126 of dosing device 120 into the appropriate liquid, and presses “LOAD.” In response to the “LOAD” command, dosing device 120 loads, or aspirates, a volume of the pharmaceutical liquid from liquid source 127 that corresponds to the dose information. However, after dosing device 120 loads the liquid, it does not dose the rodent. Instead, it signals dosing device controller 100 via second link 110 that it has loaded the liquid, and waits for further instructions.

When dosing device controller 100 receives a signal from dosing device 120 that the appropriate dose is loaded, controller 100 displays “dose loaded” or another appropriate signal to the user on display 115, which lets the user know that the dose is loaded. Then the user confirms that the rodent is prepared for dosing. This is useful because the rodent may need to be positioned in a particular way in order to be properly dosed. For example, in order to properly dose the rodent by way of oral gavage the user may need to insert gavage tip 126 into the rodent's stomach.

When the rodent is positioned properly for dosing, the user presses the “DOSE” button on keypad 125. This signals to dosing device 120 to deliver the pharmaceutical liquid to the rodent. Preferably the time between when the user presses the “LOAD” and “DOSE” buttons, e.g., the residence time of the liquid in dosing device 120, is short enough that the liquid does not interact or react with any materials it contacts in device 120. In some embodiments, this time is 30 seconds or less.

The user must press “DOSE” continually in order for dosing device 120 to deliver the liquid, until it delivers all of the liquid. This is useful because, for example, the rodent might not remain in a suitable position for dosing during the duration of the delivery. For example the gavage tip might slip out of the rodent's stomach. In this case, the user could easily release the “DOSE” button, reposition the rodent, and then repress the “DOSE” button in order to resume liquid delivery. Alternately, there could be a hardware problem that prevents the rodent from being dosed properly. The user could release the “DOSE” button, correct the problem, and repress the “DOSE” button to resume liquid delivery. This feature can be useful for minimizing the effect of unavoidable errors.

During and after the delivery of the liquid, dosing device 120 sends signals to controller 100 via link 110. For example, dosing device 120 sends signals corresponding to the pressing and releasing of keys on keypad 125, and/or for the duration and velocity of loading and delivery of the pharmaceutical liquid. Signals from dosing device 120 allow controller 100 to monitor the progress and result of the delivery, which is used to construct a delivery history.

Controller storage medium 160 locally stores a history, or audit trail, of the liquid delivery. For example this history can include the time of the dosing, the actual volume of liquid delivered, the rate of delivery, the number and duration of delivery interruptions, the outcome of the delivery, and information about any hardware errors that may have occurred. Controller 100 includes a real-time clock, and records a time stamp for each event in the history. After the delivery is completed, dosing device controller 100 automatically transfers the history stored on controller storage medium 160 to database terminal 140, via link 130. Then database terminal 140 stores the information on database 150.

It can be useful to maintain a detailed dosing history for each dosing event for each rodent. The history can be used, for example, to study toxicological effects of the pharmaceutical liquid over time on each rodent in the plurality of rodents. The history can also be useful in identifying data that corresponds to rodents that received anomalous or incorrect doses. In some embodiments, after database 150 stores the delivery history, or audit trail, database 150 automatically prevents the history from being altered in any way, e.g., the data is non-erasable. This can help to prevent entities either internal or external to the study from manipulating the audit trail. This can be useful at least to help ensure the accuracy of the study of the toxicological effect of the liquid on rodents, and also to help ensure that if study results are presented to a regulatory agency, that the agency should not be able to question whether the results were tampered with.

After a rodent is dosed according the protocol data stored on database 150, and the delivery history is stored on database 150, another rodent may be selected and dosed using the above-described procedure. Because each rodent has unique protocol data, the volume, concentration, and/or the identity of the pharmaceutical liquid to be delivered to the new rodent may be substantially different from that of the prior rodent. In general, the user does not need to know the value of any of these variables in order to dose the rodent with the correct amount of liquid.

The user's role is relatively limited in the operation of system 200, allowing for the rapid successive dosing of a plurality of rodents with high accuracy. The user's role generally includes obtaining a designated rodent; inserts gavage tip 126 into the liquid and pressing “LOAD;” and positioning the rodent and pressing “DOSE.” In other words, the user's role is reduced to removing the correct rodent from the cage, and aspirating and gavaging the dosage. This limited user involvement is useful because it minimizes the burden on the user, and minimizes the possibility of human error leading to the rodent receiving an incorrect dose. The user does not need to know the identity of the liquid, or how much needs to be delivered. In one embodiment of the system, it was found that actual administered doses were, on average, accurate to within about 0.5% of the desired dose for a wide range of dose volumes.

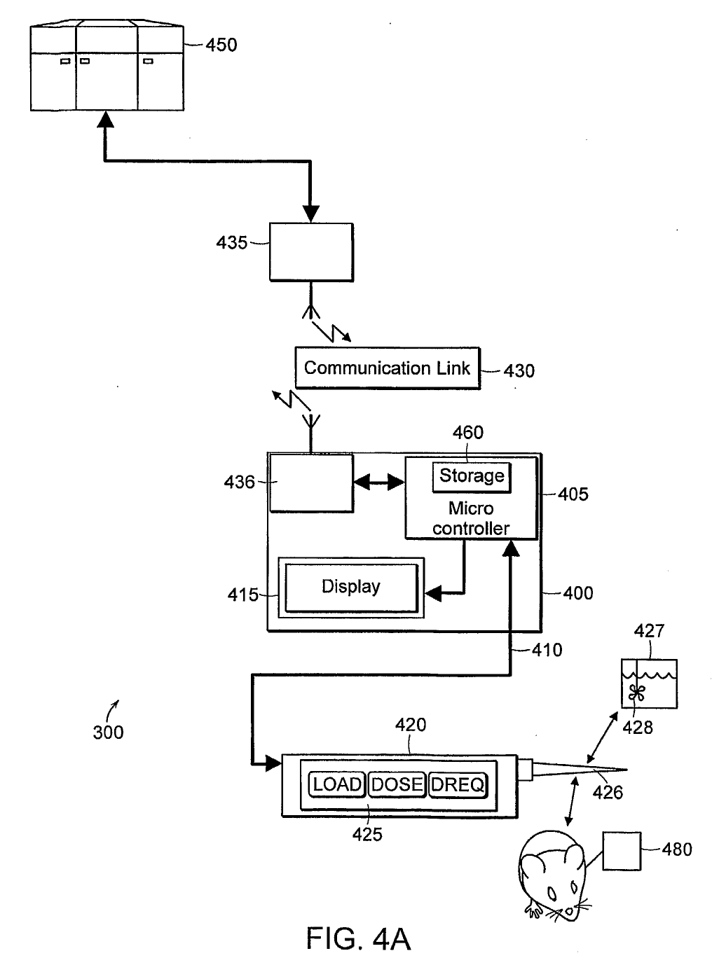

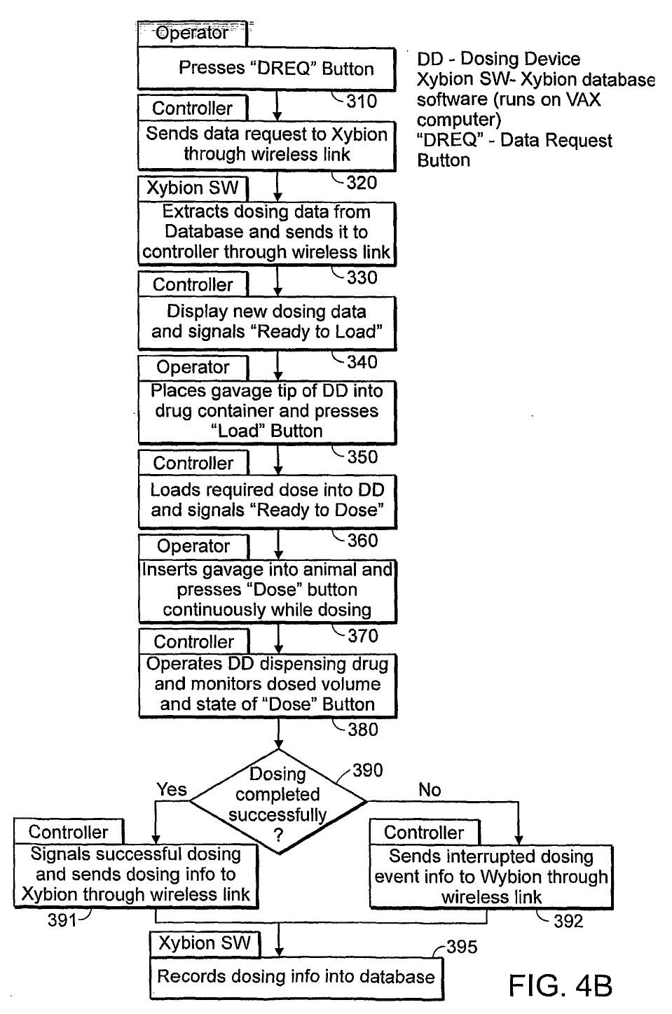

FIG. 4A illustrates another embodiment of a system for automatically delivering a dose of a pharmaceutical liquid to each rodent of a plurality of rodents. System 300 illustrated in FIG. 4A includes rodent identifier 480, database 450, dosing device controller 400, and dosing device 420. As for dosing device controller 100 of FIG. 2, dosing device controller 400 includes microcontroller 405, controller storage medium 460, display 415, and communication hardware 436. As for dosing device 120 of FIG. 2, dosing device 420 includes gavage tip 426, through which pharmaceutical liquid is aspirated and/or delivered, and keypad 425, through which a user (not shown) inputs simple commands to system 300. Controller 400 communicates with dosing device 420 via communication link 410. The different components of the system may have similar functionality to the corresponding components as described above with regard to FIG. 2.

In this embodiment, as for database 150 of FIG. 2, database 450 contains protocol data associated with identifier 480, which can be used to calculate dose information for each rodent. However in this case database 450 is in direct communication with communications hardware 435, with which it communicates with dosing device controller 400 over communications link 430. This feature makes system 300 somewhat simpler than system 200 of FIG. 2, in which database 150 communicates with dosing device controller 100 via terminal 140, optional communication controller 137, communication hardware 135, and communication link 130.

In general, database 450 and dosing device controller 400 have the additional functionality of being able to directly understand information sent from each other, because no additional hardware (e.g., communication controller 137 of FIG. 2) mediates the communication. In other words, controller 400 more or less directly accesses data on database 450, and database 450 more or less directly provides that data to controller 400.

FIG. 4B is a flow chart of the steps in the operation of system 300 of FIG. 4A, according to one embodiment of the invention. First, the user (operator) presses the “DREQ” button on keypad 427 of dosing device 420 (step 310). This signals to dosing device controller 400 to request protocol data for a rodent from database 450. In response, dosing device controller 400 sends a request to database 450, e.g., Xybion, through link 430, e.g., wireless link (step 320), for protocol data for a rodent.

Next, software running on database 450, e.g., Xybion software (SW), extracts protocol data from database 450, derives dose information (dosing data) from the protocol data, and sends the dosing data to controller 400 via link 430, e.g., wireless link (step 330). Next, controller 400 displays at least a portion of the dosing data to the operator on display 415, including identifier 480 of the rodent to be dosed (step 340). The operator then loads dosing device (DD) 420 by placing gavage tip 426 into pharmaceutical liquid source 427 and pressing the “LOAD” button on keypad 425 (step 350). Controller 400 responds by loading the liquid into gavage tip 426, and displaying “Ready to Dose” on display 415 (step 360).

Next, the operator finds the rodent associated with identifier 480, and inserts gavage tip 426 into the rodent's stomach. Then, the operator continuously presses the “DOSE” button on keypad 425 (step 370). In response, dosing device (DD) 420 delivers the liquid to the rodent, and controller 400 monitors signals from dosing device 420 in order to determine the volume and status of the delivery (step 380).

After the liquid delivery is complete, controller 400 assesses whether the liquid was successfully delivered to the rodent (step 390). If the liquid was delivered without interruption, controller 400 sends a delivery history to database 450, e.g., Xybion database, indicating that the delivery was successful (step 391). If the liquid was delivered with interruption, controller 400 sends a delivery history to database 450, e.g., Xybion database, indicating that the delivery was interrupted (step 392). Then database software, e.g., Xybion software (SW), records the delivery history (dosing info) to database 450.

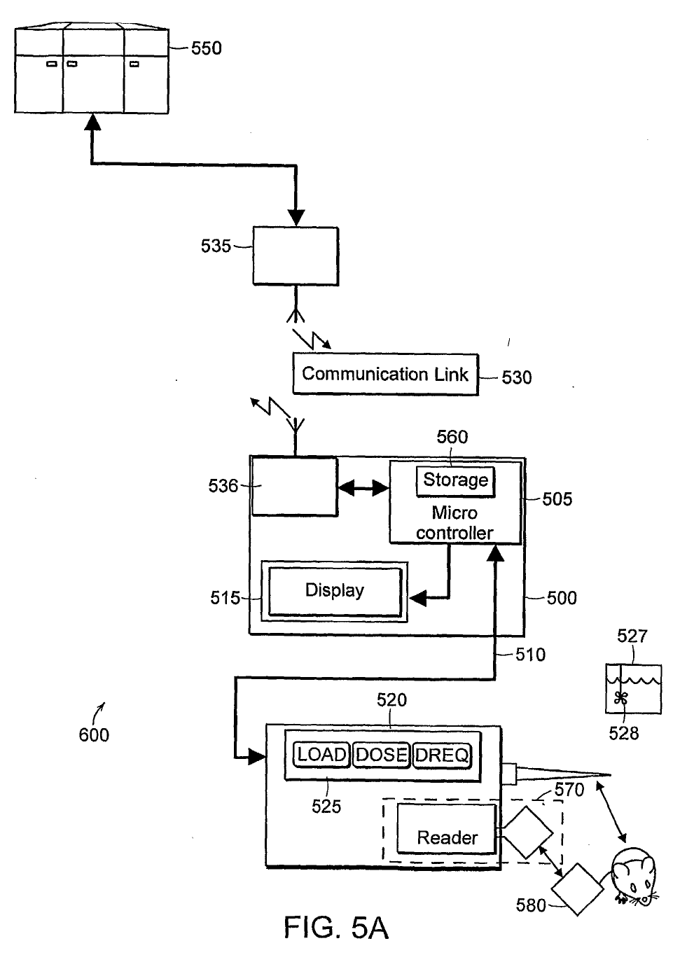

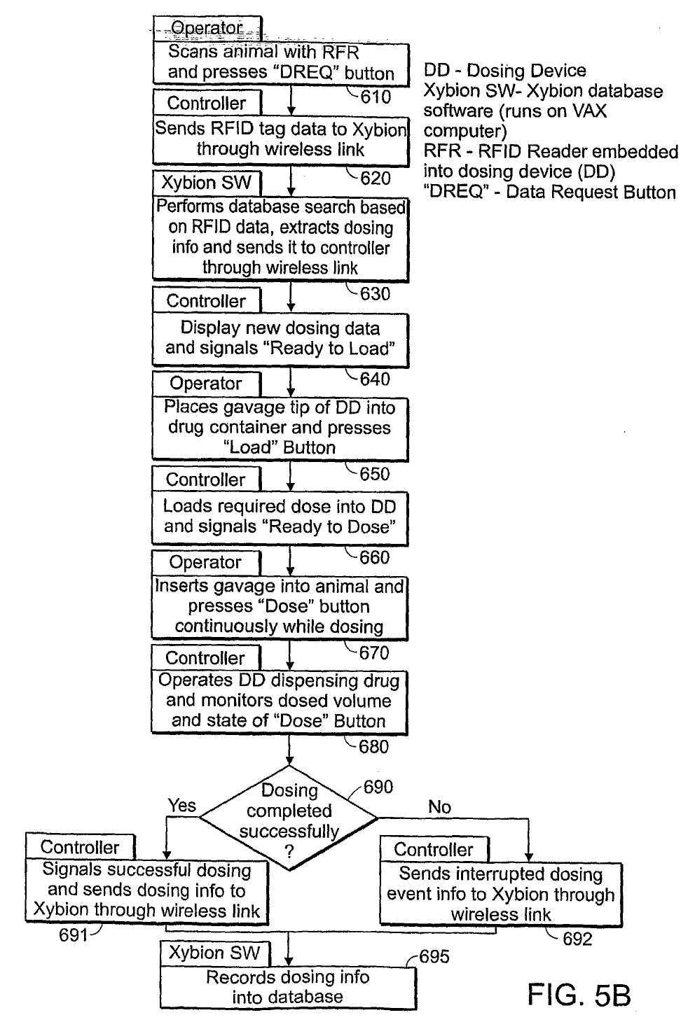

FIG. 5A illustrates another embodiment of a system for automatically delivering a dose of a pharmaceutical liquid to each rodent of a plurality of rodents. System 600 illustrated in FIG. 5A includes rodent identifier 580, database 550, dosing device controller 500, and dosing device 520. As for dosing device controller 100 of FIG. 2, dosing device controller 500 includes microcontroller 505, controller storage medium 560, display 515, and communication hardware 536. As for dosing device 120 of FIG. 2, dosing device 520 includes gavage tip 526, through which pharmaceutical liquid is aspirated and/or delivered, and keypad 525, through which a user (not shown) inputs simple commands to system 300. Controller 500 communicates with dosing device 520 via communication link 510. The different components of the system may have similar functionality to the corresponding components as described above with regard to FIG. 2.

In this embodiment, as for database 150 of FIG. 2, database 550 contains protocol data associated with identifier 580, which can be used to calculate dose information for each rodent. However in this case database 550 is in direct communication with communications hardware 535, with which it communicates with dosing device controller 500 over communications link 530. This feature makes system 500 somewhat simpler than system 200 of FIG. 2, in which database 150 communicates with dosing device controller 100 via terminal 140, optional communication controller 137, communication hardware 135, and communication link 130.

In general, database 550 and dosing device controller 500 have the additional functionality of being able to directly understand information sent from each other, because no additional hardware (e.g., communication controller 137 of FIG. 2) mediates the communication. In other words, controller 500 more or less directly accesses data on database 550, and database 550 more or less directly provides that data to controller 500.

In this embodiment, in contrast to the above-described embodiments, database 550 does not direct the user as to which rodent is to be dosed. Instead, the user uses identifier reader hardware 570 to obtain rodent identifier 580 for a rodent of the user's choosing. Then, controller 500 uses rodent identifier 580 to access protocol data stored on database 550 that is associated with the associated rodent. This is useful because the user does not need to spend time looking for a particular rodent, as is the case in the systems illustrated in FIGS. 2 and 4A. In essence, the rodents are “self-identifying.”

In one embodiment, identifier 580 is a value associated with an electronically readable tag. For example, identifier 580 can be a value associated with a radio frequency identification (RFID) tag implanted in or attached to the rodent. Implantable RFID tags are commercially available. Alternately, identifier 580 can be a number that is encoded by a bar code, which could be printed on a tag attached to the rodent or to the rodent cage, or even tattooed on the rodent. In another embodiment, identifier 580 is a number or other distinguishing mark or symbol that is printed on a tag attached to the rodent or to the rodent cage, or even tattooed on the rodent. In general, identifier 580 has the feature of being readable by suitable hardware, e.g., identifier reader hardware 570.

Dosing device 520 includes identifier reader hardware 570, which enables system 500 to obtain rodent identifier 580 with the assistance of the user. Identifier reader hardware 570 is hardware that is appropriate to the particular embodiment of identifier 580. For example, if identifier 580 is encoded in a bar code, then identifier reader hardware 570 could be a bar code reader. If identifier 580 is stored in an RFID tag, then hardware 570 could be an RFID reader. If identifier 580 is a number or other distinguishing mark or symbol tattooed on the rodent, then hardware 570 could be for example an optical reader. Hardware 570 could even be a keypad through which a user can manually enter the identifier. In one embodiment, hardware 570 is a touch screen on display 515 through which the user can manually enter the identifier. In general, identifier reader hardware 570 has the feature of electronically obtaining identifier 580 by suitable means.

FIG. 5B is a flow chart of the steps in the operation of system 600 of FIG. 5A, according to one embodiment of the invention. First, the user (operator) selects and scans a rodent with identifier reader hardware 570, e.g., RFID Reader (RFR) to obtain rodent identifier 580, e.g., a value associated with an RFID tag implanted in the rodent. Then the user presses the “DREQ” button on keypad 527 of dosing device 520 (step 610). This signals to dosing device controller 500 to request protocol data for the operator-selected rodent from database 550. In response, dosing device controller 500 sends a request to database 550, e.g., Xybion, through link 530, e.g., wireless link (step 620), for protocol data for the rodent associated with scanned identifier 580.

Next, software running on database 550, e.g., Xybion software (SW), extracts protocol data from database 550 for the rodent associated with identifier 580, derives dose information (dosing data) from the protocol data, and sends the dosing data to controller 500 via link 530, e.g., wireless link (step 630). Next, controller 400 displays at least a portion of the dosing data to the operator on display 515, including identifier 580 of the rodent to be dosed (step 640). In this case the displayed identifier should match the identifier 580 of the rodent that the operator scanned. The operator then loads dosing device (DD) 520 by placing gavage tip 526 into pharmaceutical liquid source 527 and pressing the “LOAD” button on keypad 525 (step 650). Controller 500 responds by loading the liquid into gavage tip 526, and displaying “Ready to Dose” on display 515 (step 660).

Next, the operator inserts gavage tip 526 into the rodent's stomach. Then, the operator continuously presses the “DOSE” button on keypad 525 (step 670). In response, dosing device (DD) 520 delivers the liquid to the rodent, and controller 500 monitors signals from dosing device 520 in order to determine the volume and status of the delivery (step 680).

After the liquid delivery is complete, controller 500 assesses whether the liquid was successfully delivered to the rodent (step 690). If the liquid was delivered without interruption, controller 600 sends a delivery history to database 550, e.g., Xybion database, indicating that the delivery was successful (step 691). If the liquid was delivered with interruption, controller 500 sends a delivery history to database 550, e.g., Xybion database, indicating that the delivery was interrupted (step 692). Then database software, e.g., Xybion software (SW), records the delivery history (dosing info) to database 650.

In general, the database in the above-described embodiments need not be a centralized, remote database at all, but can be any storage device or combination of storage devices that can store relevant rodent information, protocol data and/or dose information, and also store a detailed audit trail of the delivery of liquid. For example, the storage device can be co-located with the controller, dosing device, and/or to the rodent itself. For example, the storage device could be a hard drive housed with the controller, from which the controller can determine dose information for the rodent, and to which the controller can record a delivery history. Or, for example, the storage device can be distributed, with more than one different local or remote device storing different kinds of relevant information. For example, a rodent information storage device, e.g., hard drive, could store rodent information, a protocol storage device, e.g., hard drive, could store protocol data, and an audit data storage device, e.g., computer, could store an audit trail, e.g., delivery history. The different storage devices could each be located locally to or remotely from the system. In general, the storage device or devices preferably include robust data protection mechanisms and/or software that prevents tampering with the data.

For example, in one embodiment, referring to FIG. 5A, rodent identifier 580 is a readable/writable RFID tag associated with, e.g., attached to or implanted in, the rodent. This tag stores protocol data for the rodent. This tag can also store, for example, the rodent's weight, surface area, the results of any analytical tests performed on the rodent, and the dosing history of the rodent. In other words, the tag has the functionality of a database. When the user scans identifier 580 with identifier reader hardware 570, e.g., an RFID reader, the protocol data for that rodent is transmitted to dosing device controller 500. Then controller 500 derives dose information from the protocol data, and with input from the user on keypad 525, dosing device 520 delivers liquid to the rodent. Alternately, the tag itself calculates or stores the dose information for the rodent, which controller 500 then uses directly to instruct dosing device 520 as to the correct dose. In general, rodent identifier 580 does not need to be a readable/writable RFID tag, but can be any small device that can be attached to or implanted in the rodent, which can store protocol data and a delivery history for the rodent.

Controller 500 monitors the delivery of liquid by dosing device 520 and constructs a delivery history, or audit trail, as described above. Then, instead of sending the delivery history to database 550, controller 500 sends the delivery history to readable/writable RFID tag, which stores the history. Optionally, the readable/writable RFID tag is a “write once” device, in which the delivery history cannot be altered after it is recorded to the tag.

Further, dosing device 520 could be physically integrated with controller 500. In other words, 520, 500 could be a single device with the functionality of receiving protocol data or dose information from rodent identifier 580, automatically dosing the rodent with a calculated amount of liquid, and constructing a delivery history and sending that history to rodent identifier 580. In this case, the system would include just two components.

In some embodiments, the controller and database exchange information that is encoded in strings of ASCII characters. Different pieces of information sent together in the strings can be concatenated together with a delimiter such as a <TAB>. Additionally, units associated with the pieces of information can also be included by including a <SPACE> between the pieces of information and the associated units. For example a string that encodes a rodent identifier (A1001), a rodent weight (31.4 g), a dosing volume (20.0 ml), and a dosing concentration (mg/kg) can be:

A1001<TAB>31.4<SPACE>g<TAB>20.0<SPACE>ml<TAB>3.2<SPACE>mg/kg

The units of the rodent weight, dose volume, and dosing concentration can be set in general to any appropriate units. The controller and database can also be programmed to understand any unit of mass or volume that is input. In general however any format that allows the appropriate information to be communicated between and interpreted by the controller and the database. The information exchanged between the controller and the dosing device can take a similar format. In general, the dosing device does not use information about the rodent identifier, but can use appropriately encoded information about the dose.

While dosing is provided by oral gavage in the described embodiments, any desired method of dosing the rodent can be implemented. For example, nasal gavage, intravenous injection, subcutaneous injection, intramuscular injection, or peritoneal injection could be used. Depending on the desired dosing method, an appropriate dosing device would be implemented in such a way that it can be electronically controlled. In general the rodent need not be dosed with a pharmaceutical liquid at all. The rodent could be dosed with any composition that can be automatically administered to the rodent by appropriate means.

In general, the controller does not need to store the delivery history on a controller storage medium, but could instead transmit a delivery history directly to the database.

In some embodiments, the protocol data may require the rodent to receive two or more pharmaceutical liquids. In this case, appropriate information would be provided to the user via the display on the dosing device controller. For example, information about the identity of the first liquid would be displayed, and the user would subsequently aspirate and deliver the first liquid to the rodent. Then, information about the identity of the second liquid would be displayed, and the user would subsequently aspirate and deliver the second liquid to the rodent.

In general, the animals with which the described embodiments can be used do not have to be rodents, or even mammals. The described embodiments can be used with any kind of animals, even humans, that can be dosed with controlled amounts of pharmaceutical compositions.

While there have been shown and described examples of the present invention, it will be readily apparent to those skilled in the art that various changes and modifications may be made therein without departing from the scope of the invention as defined by the appended claims. Accordingly, the invention is limited only by the following claims and equivalents thereto.

What is claimed is:

1. A method of automatically delivering a dose of pharmaceutical liquid to an animal, the method comprising:
(a) under electronic control, automatically obtaining dose information for the animal;
(b) under electronic control, delivering a volume of pharmaceutical liquid to the animal in response to the obtained dose information; and
(c) under electronic control, automatically recording a detailed audit trail of the liquid delivery to the animal.

2. The method of claim 1, wherein the detailed audit trail includes information about at least one of a time of liquid delivery, an actual volume of liquid delivered, an interruption in the delivery, an error in the delivery, an identifier for the animal, and an outcome of the delivery.

3. The method of claim 1, wherein the audit trail is recorded in non-erasable memory.

4. The method of claim 1, wherein the dose information for the animal is a function of at least one of a concentration of the pharmaceutical liquid and a volume of the pharmaceutical liquid.

5. The method of claim 1, wherein the dose information for the animal is a function of a stored value of at least one physical parameter of the animal.

6. The method of claim 5, wherein a value of at least one physical parameter of the animal changes over time, and further comprising under electronic control updating the stored value of the at least one physical parameter over time.

7. The method of claim 5, wherein a value of at least one physical parameter comprises a weight of the animal, a surface area of the animal, a value of a test result on the animal, a change in a value of a test result on the animal, and a dose group of the animal.

8. The method of claim 5, wherein the value of the test result is a concentration of a compound in at least one of the animal's urine, hair, feces, and blood.

9. The method of claim 5, wherein the value of the test result is at least one of a size of a tumor of the animal and a size of an organ of the animal.

10. The method of claim 5, wherein the value of the test result is a measure of cell function.

11. The method of claim 10, wherein the measure of cell function is one or cell number, cell membrane integrity, enzyme activity, cell aggregation, and clotting function.

12. The method of claim 1, further comprising obtaining an identifier for the animal before delivering liquid to the animal.

13. The method of claim 1, wherein delivering a volume of pharmaceutical liquid to the animal comprises delivering the liquid by one of oral gavage, nasal gavage, intravenous injection, subcutaneous injection, intramuscular injection, and peritoneal injection.

14. The method of claim 1, wherein (a), (b), and (c) are repeated for each animal of a plurality of animals and the delivered volume of liquid is specific to each animal of the plurality of animals.

15. The method of claim 1, further comprising under electronic control loading the volume of liquid and delivering said volume of liquid to the animal.

16. The method of claim 1, further comprising under electronic control loading a first volume of pharmaceutical liquid, under electronic control delivering from said first volume of liquid a volume of liquid to the animal in response to the obtained dose information, and under electronic control monitoring an undelivered volume of liquid.

17. The method of claim 16, further comprising repeating for each animal of a plurality of animals (a), (b), and (c), wherein the volume of pharmaceutical liquid delivered to each animal is from said undelivered volume of liquid, and further comprising repeating for the animal under electronic control monitoring an undelivered volume of liquid.

18. The method of claim 16, further comprising under electronic control loading a second volume of liquid when electronic control determines whether the undelivered volume of liquid is less than the volume of liquid to deliver to a next animal.

19. The method of claim 1, wherein a device for delivering the volume of pharmaceutical liquid has a linear actuator, and wherein a motion of the linear actuator defines the delivered volume of pharmaceutical liquid.

20. The method of claim 19, wherein the electronic control uses dose information to form an instruction for moving the linear actuator by a controlled amount to deliver the volume of pharmaceutical liquid.

21. A method of automatically delivering a dose of pharmaceutical liquid to an animal, the method comprising:
(a) under electronic control, automatically calculating dose information for the animal, wherein the dose information is a dynamic function of a stored value of at least one physical parameter of the animal; and
(b) under electronic control, delivering a volume of pharmaceutical liquid to the animal in accordance with the dose information.

22. The method of claim 21, further comprising under electronic control automatically recording a detailed audit trail of the pharmaceutical liquid delivery to the animal.

23. The method of claim 21, wherein a value of at least one physical parameter of the animal changes over time, and further comprising (c) under electronic control updating the stored value of the at least one physical parameter over time.

24. The method of claim 23, further comprising repeating for the animal (a) and (b), wherein the dose information in (a) is a dynamic function of the updated stored value of the at least one physical parameter in (c).

25. The method of claim 21, wherein a value of at least one physical parameter is a function of at least one previously delivered dose of pharmaceutical liquid.

26. The method of claim 21, wherein a value of at least one physical parameter comprises a weight of the animal, a surface area of the animal, a value of a test result on the animal, a change in a value of a test result on the animal, and a dose group of the animal.

27. The method of claim 26, wherein the value of the test result is a concentration of a chemical in at least one of the animal's urine, hair, feces, and blood.

28. The method of claim 26, wherein the value of the test result is at least one of a size of a tumor of the animal and a size of an organ of the animal.

29. The method of claim 26, wherein the value of the test result is a measure of cell function.

30. The method of claim 29, wherein the measure of cell function is one or cell number, cell membrane integrity, enzyme activity, cell aggregation, and clotting function.

31. The method of claim 21, wherein a device for delivering the volume of pharmaceutical liquid includes a linear actuator, and wherein a motion of the linear actuator defines a delivered volume of pharmaceutical liquid.

32. The method of claim 31, wherein the dose information comprises an instruction for moving the linear actuator by a controlled amount in order to define a controlled volume of pharmaceutical liquid.

33. A system for automatically delivering a dose of pharmaceutical liquid to an animal, the system comprising:
storage for storing dose information for the animal and a detailed audit trail of delivery of liquid to the animal;
an electronically-controllable dosing device responsive to controller commands for delivering a volume of pharmaceutical liquid to an animal; and
a controller in communication with the storage and with the dosing device, the controller including electronic logic to:
access the storage to receive dose information;
automatically determine the volume of pharmaceutical liquid to deliver to the animal from the dose information;
automatically send controller commands to the dosing device; and
automatically send a detailed audit trail to the storage for storage.

34. The system of claim 33, further comprising electronic logic to automatically determine the dose information using a value of at least one physical parameter of the animal.

35. The system of claim 34, wherein a value of at least one physical parameter of the animal includes a weight of the animal, a surface area of the animal, a value of a test result on the animal, a change in a value of a test result on the animal, and a dose group of the animal.

36. The system of claim 33, wherein the storage comprises non-erasable memory.

37. The system of claim 33, wherein the storage is remote to the controller.

38. The system of claim 33, wherein the storage is co-located with at least one of the controller and the dosing device.

39. The system of claim 33, wherein the storage is one of a mainframe computer and a personal computer.

40. The system of claim 33, wherein the storage is an RFID chip associated with the animal.

41. The system of claim 40, wherein the RFID chip is implanted in the animal.

42. The system of claim 33, wherein the dosing device comprises an automatic syringe.

43. The system of claim 33, wherein the dosing device includes one of an electronically controllable peristaltic pump and an electronically controllable positive displacement pump.

44. The system of claim 33, wherein the dosing device includes an electronically controllable linear actuator assembly.

45. The system of claim 44, wherein the controller includes one of electronic logic and embedded software for determining a travel distance for a linear actuator using the dose information.

46. The system of claim 45, wherein the travel distance is directly related to the volume of pharmaceutical liquid delivered.

47. The system of claim 33, wherein the dosing device includes electronic circuitry to receive and respond to controller commands and to send one or more delivery signals to the controller.

48. The system of claim 47, wherein the controller includes one of electronic logic and embedded software to monitor the one or more delivery signals from the dosing device and to construct an audit trail using the one or more delivery signals.

49. The system of claim 48, wherein the audit trail comprises at least one of a time of pharmaceutical liquid delivery, an actual volume of pharmaceutical liquid delivered, an interruption in the delivery, an error in the delivery, an identifier for the animal, and an outcome of the delivery.

50. The system of claim 33, wherein the dosing device comprises an input device through which a user can input at least one of the following commands to control the system: request dose information for an animal, load a volume of pharmaceutical liquid into the dosing device, and deliver a volume of pharmaceutical liquid from the dosing device to the animal.

51. The system of claim 33, wherein the controller includes a display to provide at least one of the following pieces of information to a user: a unique animal identifier, indication that the dosing device is ready to load the volume of pharmaceutical liquid, and that the dosing device has loaded the volume of pharmaceutical liquid.

52. The system of claim 33, further comprising electronic logic responsive to controller commands to: control loading of the pharmaceutical liquid into the dosing device, control delivery of the pharmaceutical liquid to the animal from the dosing device, and to automatically monitor one or more volumes of pharmaceutical liquid loaded into and delivered from the dosing device.

53. The system of claim 52, wherein the electronic logic responds to controller commands by loading a first volume of pharmaceutical liquid into the dosing device, by delivering from said first volume of liquid a volume of liquid to a first animal in accordance with dose information for said first animal, and by monitoring a first undelivered volume of pharmaceutical liquid.

54. The system of claim 53, wherein the electronic logic responds to controller commands by delivering from said undelivered volume of pharmaceutical liquid a volume of pharmaceutical liquid to a second animal in accordance with dose information for said second animal, and by monitoring a second undelivered volume of pharmaceutical liquid.

55. The system of claim 33, wherein the controller is a wearable device.

56. The system of claim 33, further comprising an animal identifier.

57. The system of claim 56, wherein the identifier is one of a number, a bar code, and an RFID tag associated with the animal.

58. The system of claim 56, wherein the dosing device further comprises a reader for the animal identifier.

59. The system of claim 33, wherein the storage and controller communicate by one of a wired and a wireless communication link.
